(19) United States
(12) Patent Application Publication
(10) Pub. No.: US 2003/0004496 A1
(43) Pub. Date: Jan. 2, 2003

(54) Urinary catheter divided into catheter sections and a catheter package

(76) Inventor: Allan Tanghoj, Kokkedal (DK)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 09/893,514

(22) Filed: Jun. 29, 2001

(51) Int. Cl. A61M 27/00

(52) U.S. Cl. 604/544

(57) ABSTRACT

A urinary catheter having at least two catheter sections defining a passage therethrough, the sections being adapted to be arranged in a first mutual configuration in which the sections together constitute a catheter having a length longer than the length of each individual section, and having a rigidity such that the entire catheter is manipulatable as one uniform catheter tube by manipulation of at least one of the sections. The urinary catheter may also be adapted to fold, collapse, bend, separate, or otherwise adjust to include an embodiment in which the length of the catheter is less than when the sections are arranged in the first mutual configuration.

### FIELD OF THE INVENTION

The present invention relates to an elongated tubular catheter member for draining the bladder.

### BACKGROUND OF THE INVENTION

Catheters for draining the bladder are increasingly used for intermittent as well as indwelling or permanent catheterisation. Typically catheters are used by patients suffering from urinary incontinence or by disabled individuals like para- or tetraplegics who may have no control permitting voluntary urination and for whom catheterisation may be the way of urinating e.g. permitting the individual to stay seated in a wheel chair or lying in bed.

Catheterisation is thus increasingly becoming a daily-life procedure significantly improving quality of life for a large group of patients.

Typically catheters are designed for one-time use and accordingly the costs for producing, packing and sterilising a catheter is an important issue. Existing catheters are made from a single piece of a continuous catheter tube. Typically the thickness of the catheter tube is constant throughout its length.

The length of the catheter enables insertion of a certain length into the urethra until urine starts to flow. At this point a certain over-length of the catheter should be available. The over-length supports for the user to firmly hold the catheter and to guide the urine to a place of disposal and to withdraw the catheter safely and without any risk of the catheter disappearing into the urethra.

Existing catheters are designed to minimise the risk of sores in the mucous membrane and to give substantially no sensation of pain during insertion. Accordingly known catheters are typically provided with a smooth and slippery surface optimised for safe and comfortable insertion into the urethra. Therefore, it may often be difficult, not least for the disabled user, to handle the catheter by manipulation of the slippery over-length.

It is important that the tubular member does not collapse or kink and thereby blocks the passage for the urine to drain through the catheter. Existing catheters are therefore typically made from a form stabile and relatively hard but still bendable tube e.g. made from PVC, PU or PE. Since the hardness of the tubes is selected relatively high with the view to avoid kinking, the catheters may collapse if they are bend with a too small radius of curvature.

Accordingly, existing catheters not only have a considerable length but they are also typically packed in an elongate condition. Therefore the existing catheters may be troublesome to handle and to bring along, not least for the individuals for whom catheterisation is a daily-life procedure.

### DESCRIPTION OF THE INVENTION

It is an object of the present invention to overcome the above described disadvantages of the known catheters by providing a kit for preparing a catheter for draining a human bladder, the kit comprising at least two catheter sections defining a passage therein, the sections being adapted to be arranged in such a mutual configuration that the passages are joined into one passage and the sections together constitute a catheter of a length larger than the length of each individual section and having such a rigidity that the entire catheter is manipulatable by manipulation of at least one of the individual sections.

Accordingly a catheter is provided which may be foldable, collapsible, bendable, separable or in any other way adapted at least for one configuration wherein the catheter can be inserted into urethra or into an artificial urinary canal and one configuration wherein the length of the catheter is reduced. As an example, the length may be reduced to a length in the range of one half, one third, one fourth or even to one fifth of the normal required length including the required over-length for manipulation of the catheter.

A rigidity of substantially the full length of the catheter should allow for manipulation of the catheter as one uniform catheter tube. Thereby, insertion of the proximal end of the catheter may be performed without touching the part of the catheter which is going to be inserted into the urethra. Preferably the catheter is provided with a bending moment defined as the product between E-modulus and moment of inertia of at least 1 MPamm⁴.

Since the proximal (inserted) end of the catheter, for male individuals, must pass prostate in a curved passage, the proximal end portion of the catheter, e.g. the first 10-50 mm., such as 20-40 mm., such as 25-35 mm, such as the first 30 mm. of the catheter may be provided with an even lower bending moment defined as the product between E-modulus and moment of inertia of less than e.g. 0.6 MPamm⁴ or even less than 0.3 MPamm⁴. Other parts of the catheter, e.g. a distal end portion where the urine is drained into the lavatory, a bag or similar place of disposal, may similarly be provided with a reduced bending moment.

The cross-sectional flow area or the hydraulic radius defined as the ratio of the cross-sectional flow area to the wetted perimeter, may be selected independently upon the length, e.g. on the basis of the size of the urethra, which size depends on the individual using the catheter. Each of the sections may have either the same cross-sectional flow area or hydraulic radius or each section may have individual cross-sectional flow areas or hydraulic radiuses. However, at least one part of one section should have a cross-sectional shape and size adapted for the size of urethra or an artificial urinary canal. Similarly one section should preferably have a length selected on the basis of the length of the urethra or the urinary canal. Thereby it may be achieved that only one section is to be inserted and therefore no transition between sections needs to be inserted. However, especially for male individuals where urethra is particularly long, a catheter having an inserted length divided in two sections or more may be provided. In this specific case it will be appropriate to provide a transition between the sections which at least on the outer surface of the catheter have substantially no recess or sharp edge.

Preferably at least one of the catheter sections is provided in a length in the range of 50-90 mm., such as in the range of 55-85 mm., such as in the range of 60-80 mm. such as with a length in the size of 70 mm. which length has been found to be a suitable insertable length for most female individuals. For male individuals, catheter sections may preferably be provided in a length in the range of 180-250 mm., such as in the range of 190-240 mm., such as in the range of 200-230 mm. such as in the size of 220 mm. For the male individuals it may further be preferred to provide at least a part of the inserted end of the catheter in a material or in dimensions so that a the tube becomes very flexible, without kinking. This will easy the passage of the catheter past prostate.

The outer cross-sectional shape of at least one of the sections should preferably be substantially circular with a cross-sectional area in the range of 0.5 mm²-30 mm².

Even more preferred is to provide at least one of the sections with a hydraulic radius (“cross-sectional area”/“circumferential length”) in the size of 0.2-1.5 mm.. Alternatively, at least one of the sections should have a cross-sectional shape matching the shape of urethra or an artificial urinary canal, still with a cross-sectional area in the range of 0.5 mm²-30 mm². or a hydraulic radius in the size of 0.2-1.5 mm. However, the other of the sections does not necessarily have to have the same cross-sectional shape, nor the same hydraulic radius. The wall thickness of the catheter should preferably be in the range between 0.5-1.5 mm.

The catheter or at least a part of the catheter could be made from a thermoplastic elatomeric material, other thermoplastic materials, curable elastomeric materials, polyamide resins or elastomers or any mixture thereof, i.e. the group may comprise materials like, PVC, PU, PE, latex, and/or Kraton™.

According to a preferred embodiment, the present invention relates to a urinary catheter divided into completely separated catheter sections. Each catheter section has at least one end provided with means for connecting the section with another section corresponding to an adjacent part of the catheter. As an example the catheter may be divided into two tubular connectable pieces connected by connecting means.

Preferably, the connecting means are provided with a rigidity allowing for manipulation of at least one of the catheter sections by manipulation of one of the other catheter sections. At least, the connection between each of the pieces should provide sufficient rigidity to allow one proximal section to be inserted into the urethra by manipulation of one of the other sections. Therefore, the connection is preferably provided so that at least the part of the catheter extending the connection zone, has a bending moment defined as the product between E-modulus and moment of inertia of at least 0.6 MPamm⁴ such as at least 1 MPamm⁴. In order not to have the individual sections falling apart during use, the connection should preferably be adapted to take up an axial force of at least 0.5 Newton or at least to take up an axial force larger than the axial force required for withdrawal of the catheter from the urethra or artificial urinary canal.

The pieces may be connected e.g. telescopically or via a hinge enabling one of two sections to rotate in relation to the other of the two sections. It is appreciated that the sections are in fixed engagement so that they do not disconnect during use of the catheter, while urine is drained through the catheter. However, since the urine is always drained in one direction the connection does not necessarily have to be liquid tight. As an example a telescopic connection may be established by inserting the section adapted for insertion into urethra into a distal section. The flow direction of the urine will at least substantially prevent the connection from leaking even though the connection as such is not completely liquid tight. However, a completely sealed connection may provide an even safer catheter with a reduced risk of contaminating hands etc.

According to another preferred embodiment, the catheter may comprise at least two sections not being separated but being divided by a bendable zone. The bendable zone could e.g. be a bellow shaped section or the zone could be an area wherein the thickness of the tubular material is smaller and wherein the zone accordingly has a lower bending moment. The zone could e.g. be provided in a more resilient or flexible material allowing for bending the catheter tube without kinking or damaging the tube.

In general, the problems of introducing a catheter into urethra depend not only of the size of the introduced part of the catheter but also on the slipperiness of the introduced part. The catheter section or at least a part of the catheter section or sections adapted for insertion into urethra or an artificial urinary canal may provide a surface slipperiness for easy and safe insertion. However, it has been found that lubricated or slippery surfaces, are difficult to handle, not least for a user having reduced dexterity. It is therefore an object of the present invention to provide a catheter with an inserted part being treated so as to provide a slippery surface and another part not being treated, so as to provide a surface which may easily be handled. The division of the catheter into one part being treated and one part not being treated may preferably follow the aforementioned division of the catheter with the purpose of making the catheter collapsible or separable. According to an alternative embodiment, the parts may be provided in the form of one part being smooth and another part being provided with a rough surface.

According to a preferred embodiment, at least one of the sections is provided with gripping means easing a firm grip in the catheter. Not least for the disabled user, the gripping means will improve the value of the catheter considerably. Gripping means may be provided as a radially extending flange or flanges or as a zone having a large outer cross sectional diameter. The catheter, or at least one of the catheter sections, may also be provided with means for engaging an external handle. As an example, one of the tubular catheter tubes may be provided with a ring-shaped bulge for attaching a handle. The ring shaped bulge could be provided as a short tubular piece of plastic with a larger radial size than the catheter, the catheter being inserted and glued into the short piece of plastic.

A section provided with a hydrophilic surface treated with a liquid swelling medium may provide an excellent lubrication for the insertion and also provide compatibility with the body tissue. It is therefore a further preferred embodiment of the invention to provide at least one of the sections with a hydrophilic surface layer.

One of the catheter sections could be used as a sterile package for the other sections, e.g. by arranging the sections in a telescopic manner inside one section, closing and sealing that section in both ends, e.g. by a peelable and optionally a metallised foil e.g. made from a thermoplastic elatomeric material, other thermoplastic materials, curable elastomeric materials, polyamide resins or elastomers or any mixture thereof, i.e. the group may comprise materials like, PVC, PU, PE, latex, and/or Kraton™, thereby allowing for sterilising the assembly by radiation.

The liquid swelling medium for the hydrophilic surface may be provided in the package for initiation of the low friction character already when the catheter is being packed. The liquid swelling medium may simply be a saline solution, a bactericidal solution capable of swelling the hydrophilic surface and capable of keeping the surface in a sterile condition or it may be pure water. The swelling may also be initiated already before packaging of the catheter, the catheter then being packed in a substantially gas impermeable package for conservation of the moistened surface. Furthermore, the liquid swelling medium may be provided in a capsule or container packed together with the catheter for swelling of the hydrophilic material immediately prior to the insertion.

According to a second aspect the present invention relates to a bendable urinary catheter for draining a human bladder comprising:

a flexible elongated tube with an inner cross-sectional shape and size defining a first conduit for draining urine, said tube having an insertion end and a discharge end, and

a supporting member being introduced into the first conduit and provided with an outer cross-sectional shape and radial size substantially equal to the inner cross-sectional shape and size of the elongate tube so as to support said tube against collapsing during bending of the tube, the supporting member having a flexibility allowing curling.

The flexible elongated tube could be a regular medical plastic hose, closed in the insertion end and provided with holes for draining the urine, the flexible elongated tube thereby having the shape of a regular catheter of the known kind. Preferably, the tube or at least a part of the tube is made from a thermoplastic elatomeric material, other thermoplastic materials, curable elastomeric materials, polyamide resins or elastomers or any mixture thereof, i.e. the group may comprise materials like, PVC, PU, PE, latex, and/or Kraton™.

The supporting member supports the catheter to avoid collapsing when the catheter is bend, e.g. for the purpose of packing the catheter in user friendly short packages. The supporting member may be either solid or the supporting member may be hollow and thus defining a second conduit. The solid supporting member should be adapted for removal prior to draining of the bladder, whereas a hollow supporting member may remain inside the tube while the bladder is emptied through the first and second conduit.

The supporting member may as an example be glued inside the elongated tube or the supporting member may be even be moulded into the tube during the process of producing the tube. The supporting member may even be completely integrated in the elongated tube.

The supporting member could be made from any suitable material such as e.g. plastic, steel, aluminium, a thermoplastic elatomeric material, other thermoplastic materials, curable elastomeric materials, polyamide resins or elastomers or any mixture thereof. As an example, the supporting member may be a spring provided in a length in the range of 20-60 mm, such as in the range of 30-50 mm., such as in the range of 35-45 mm. The spring should be positioned inside the elongated tube in the zone where it is desired to bend the catheter, e.g. midway along the longitudinal axis of the elongated tube. During use, the urine is drained through the first conduit of the elongated tube and past the supporting member through the second conduit.

According to a preferred embodiment, the supporting member is provided in a length in the range of 60-120 mm, such as in the range of 70-110 mm., such as in the range of 80-100 mm. and the supporting member may even be extending out of the discharge end of the elongated tube. This will enable the user to remove the supporting member during the process of inserting the catheter into urethra.

According to a further preferred embodiment, the supporting member is provided with gripping means for easing withdrawal of the supporting member from the discharge end during insertion of the catheter.

### DETAILED DESCRIPTION OF THE INVENTION

Preferred embodiments of the invention will now be described in details with reference to the drawing in which:

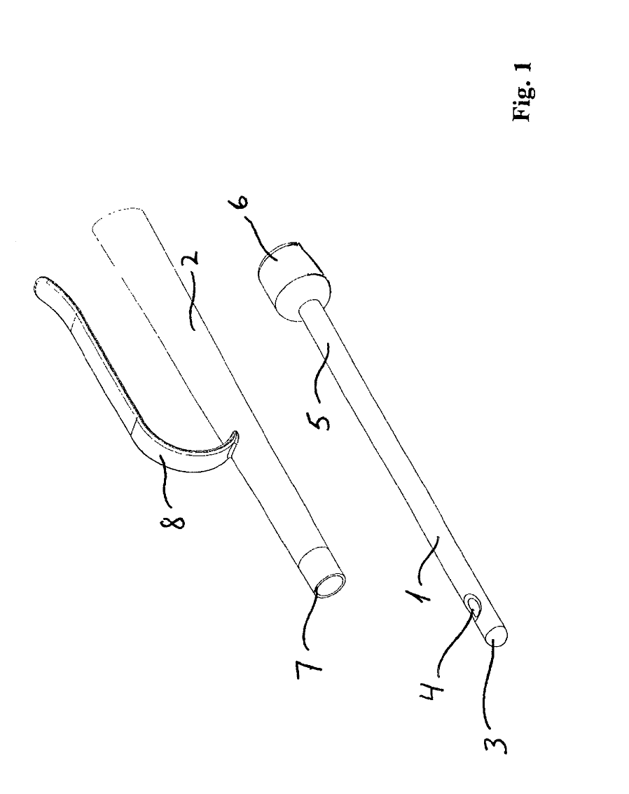

FIG. 1 shows a catheter kit according to the present invention,

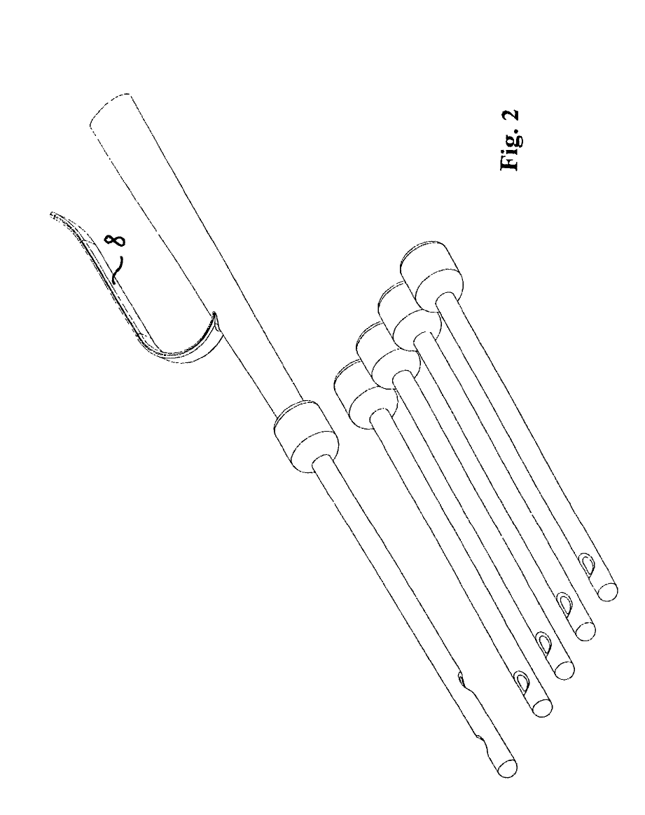

FIG. 2 shows the catheter kit of FIG. 1, assembled into a configuration for use,

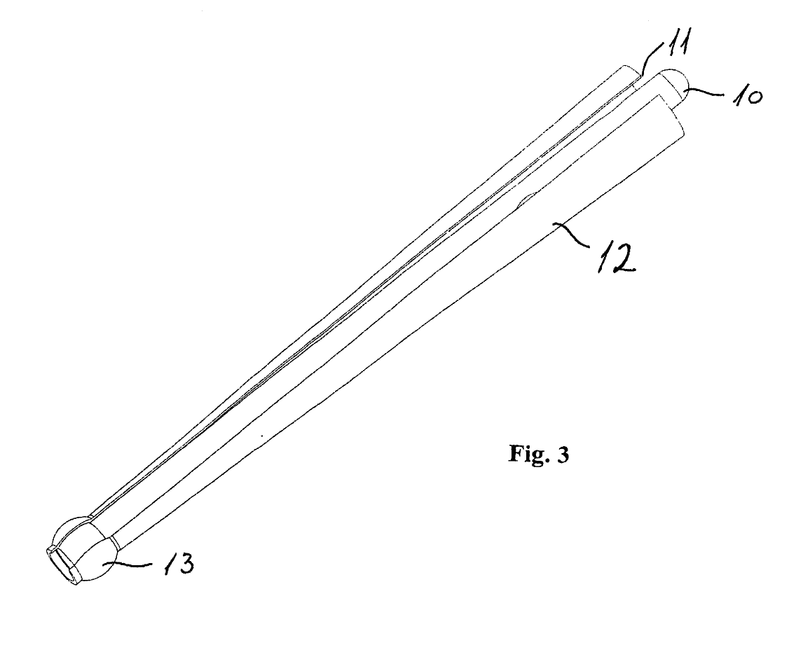

FIG. 3 shows a “Swiss-knife” embodiment of a catheter kit according to the present invention,

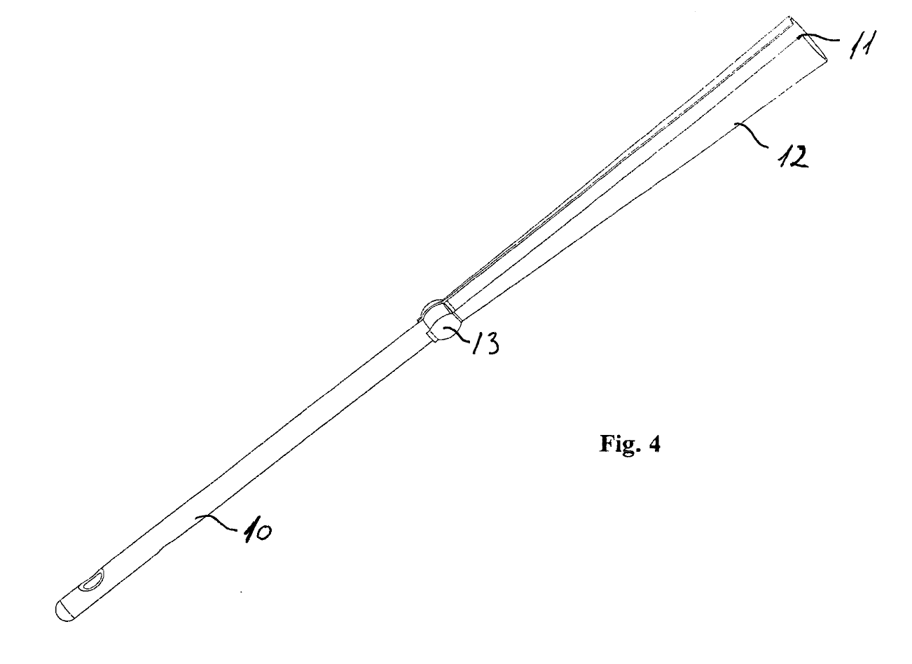

FIG. 4 shows the catheter kit of FIG. 3, unfolded and arranged in a configuration for use,

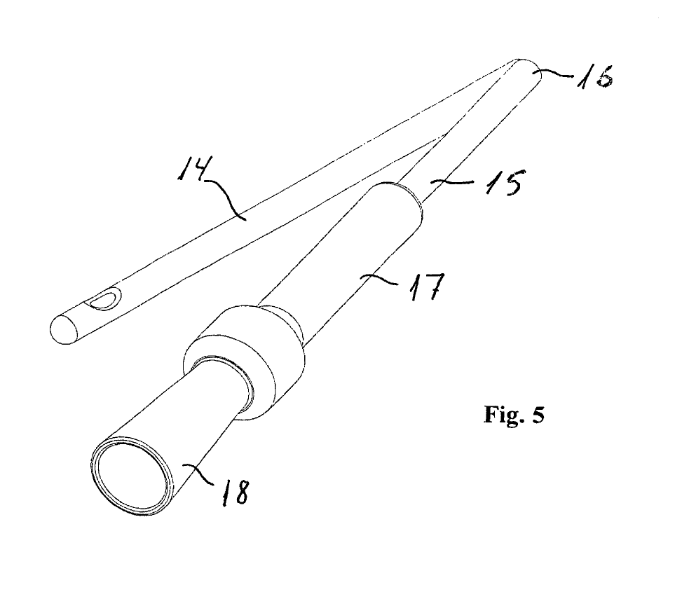

FIG. 5 shows a collapsed catheter provided with a reinforcement sleeve,

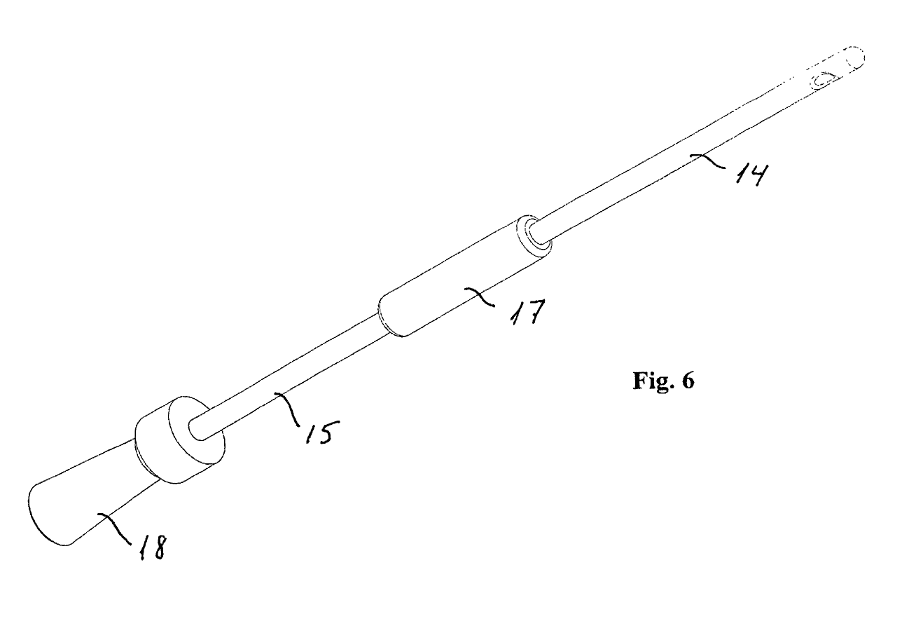

FIG. 6 shows the catheter kit of FIG. 5, unfolded and in a configuration for use,

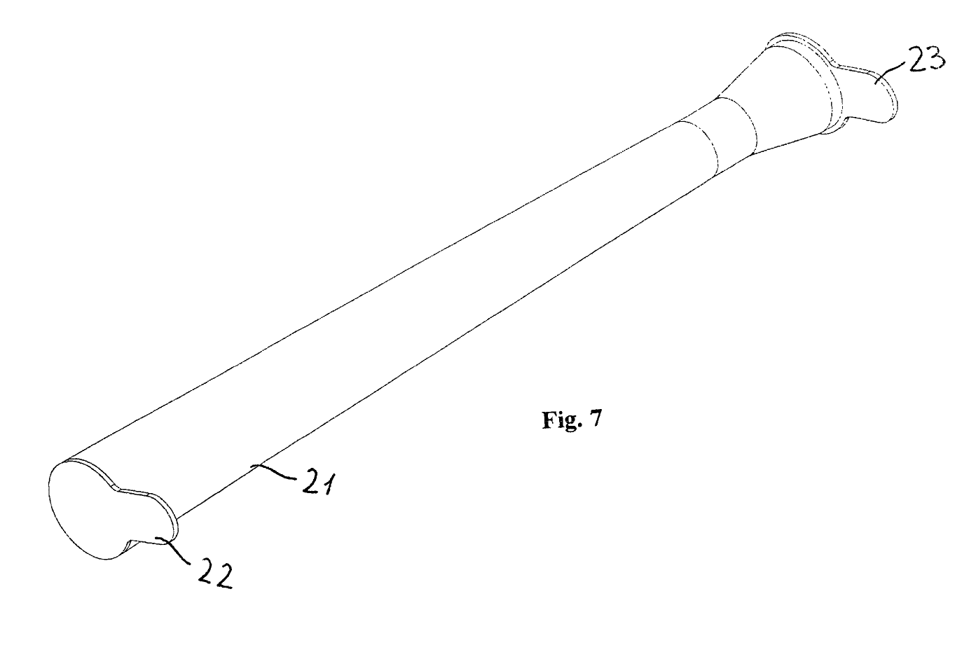

FIG. 7 shows an embodiment of the kit, wherein one catheter part is inserted for storage into another of the catheter parts thus substituting a catheter package,

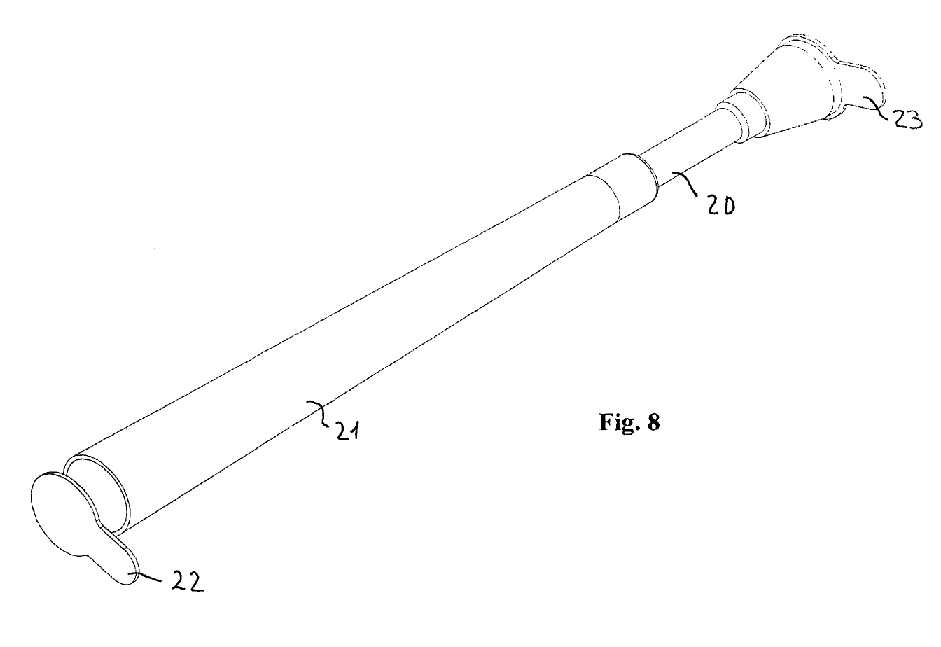

FIG. 8 shows the embodiment of FIG. 7, wherein the inserted catheter part is partially withdrawn from one end of the package,

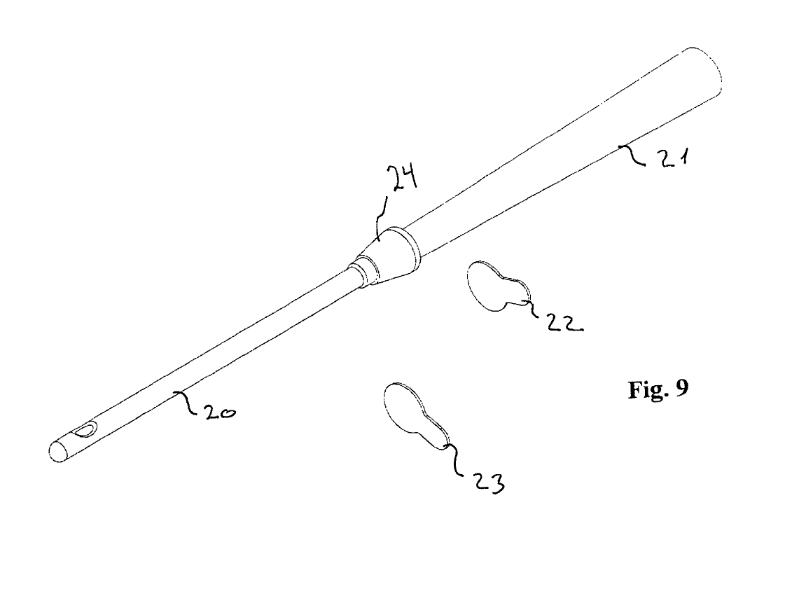

FIG. 9 shows the embodiment of FIGS. 7 and 8, wherein the inserted catheter part is completely withdrawn from the package and then attached to the other end of the package, the package thus functions as a handle for manipulation of the catheter.

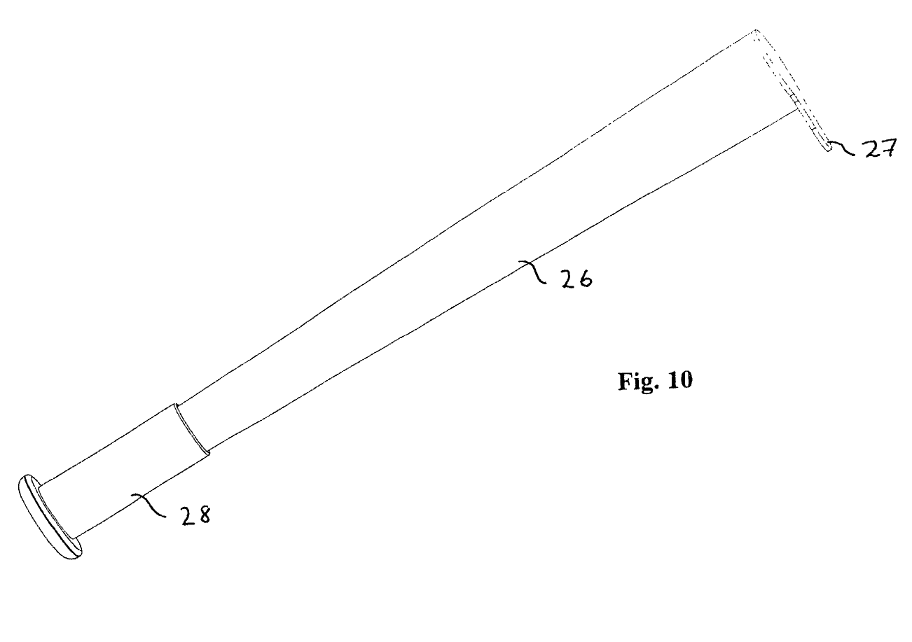

FIG. 10 shows a folded telescopic catheter kit,

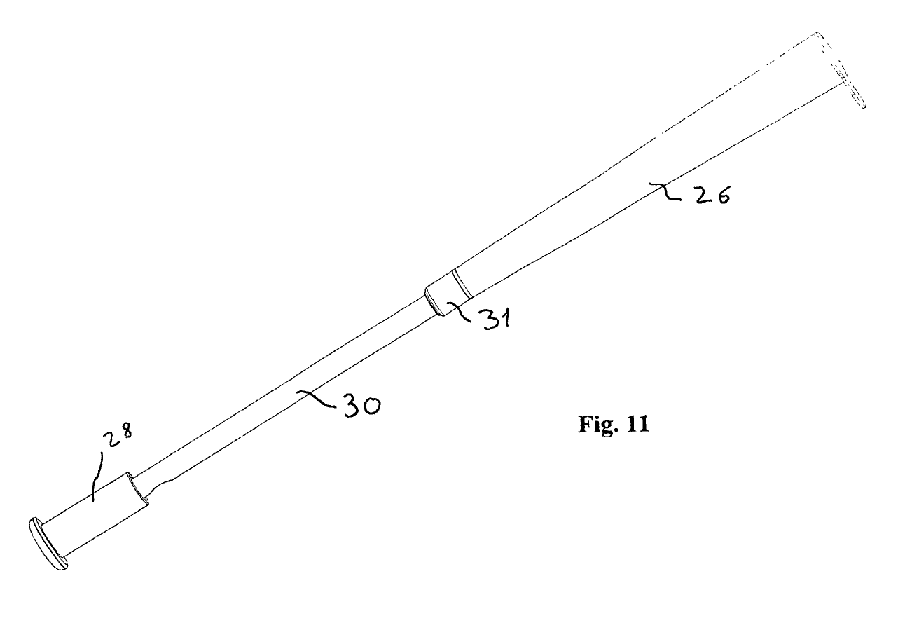

FIG. 11 shows the catheter kit of FIG. 10, in an extended configuration,

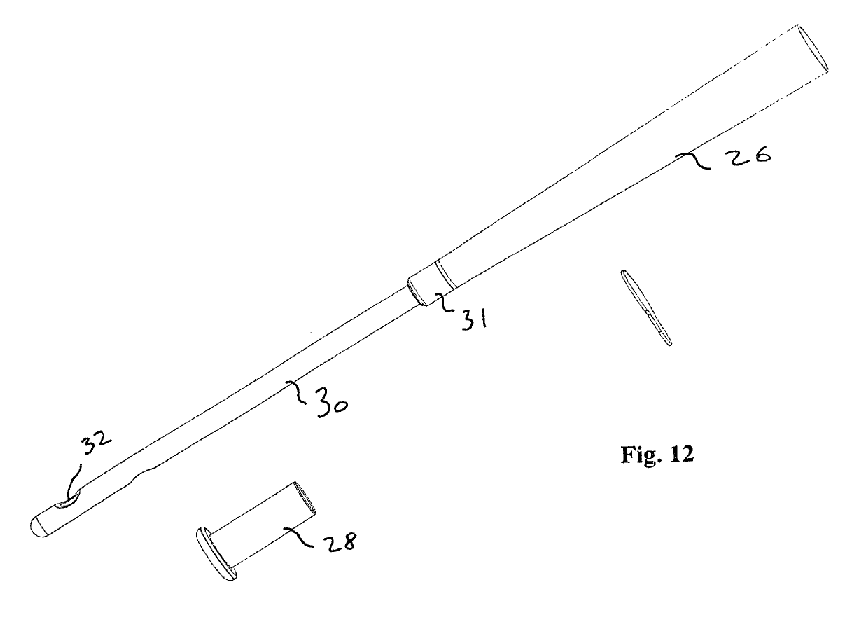

FIG. 12 shows the catheter kit of FIG. 10, unfolded and after withdrawal of the combined closure and withdrawal cap,

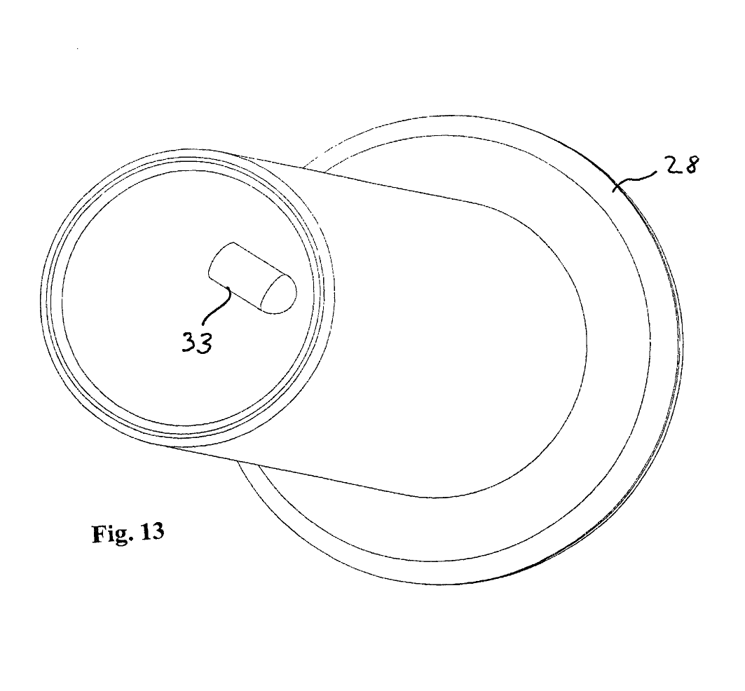

FIG. 13, shows a preferred embodiment of a combined closure and withdrawal cap for the kit shown in FIGS. 11 and 12,

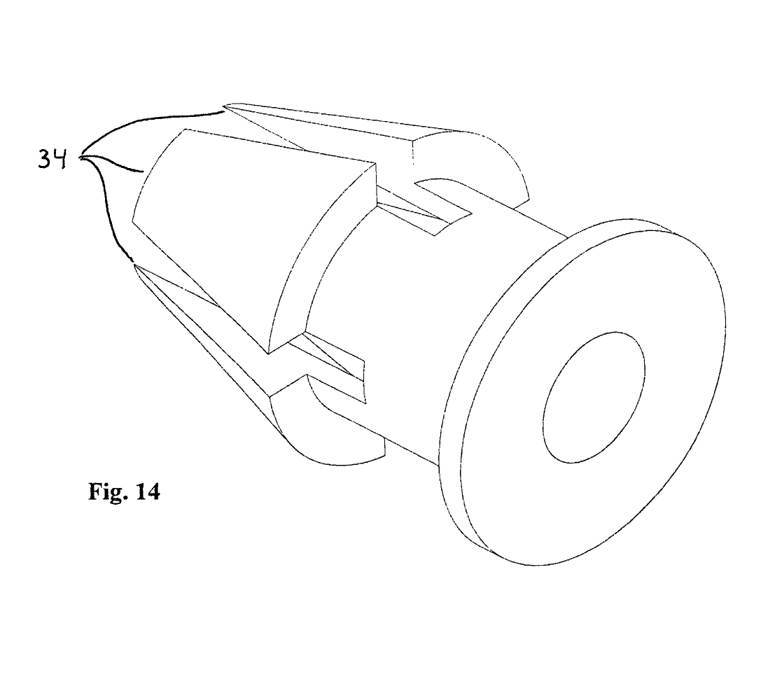

FIG. 14 shows yet another preferred embodiment of a combined closure and withdrawal cap for the kit shown in FIGS. 11 and 12,

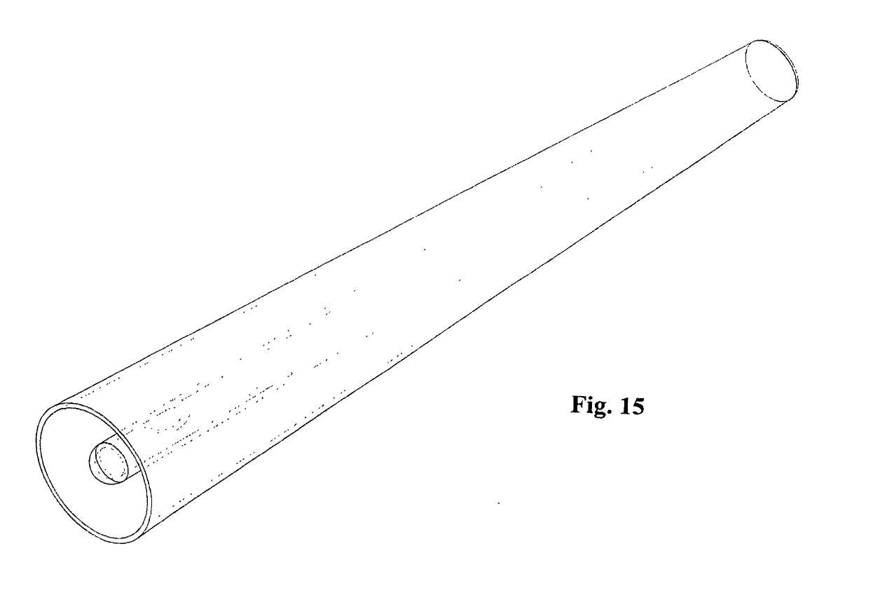

FIG. 15 shows a kit wherein a distal part of the catheter is curled over inserted part of the catheter so as to protect the inserted part of the catheter.

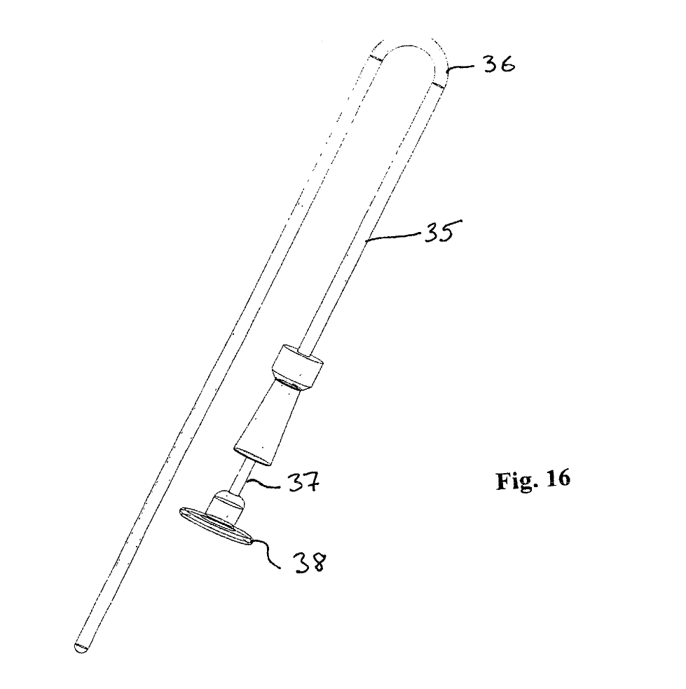

FIG. 16 shows a bendable catheter with a supporting supporting member,

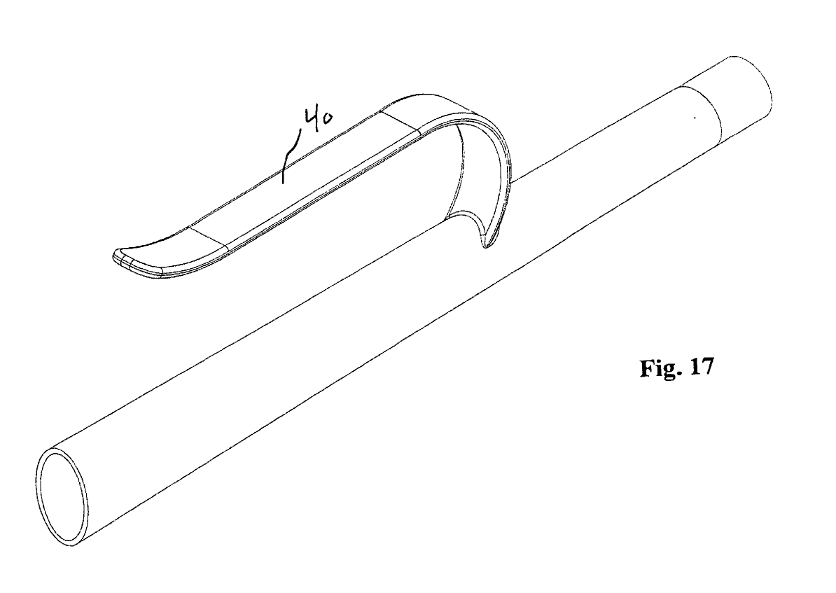

FIG. 17 shows a catheter part provided with gripping means for easing the handling of the catheter, and

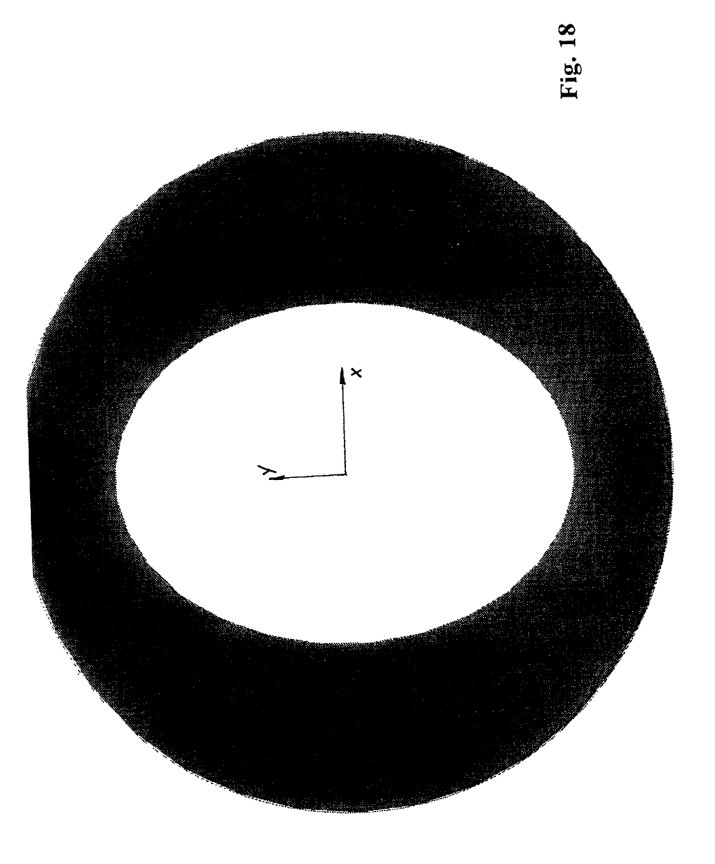

FIG. 18 shows a preferred cross-sectional shape of a catheter part adapted for insertion into the urethra.

Referring to FIG. 1, a catheter kit according to the present invention comprises a first elongate tubular catheter section 1 adapted for insertion into urethra or an artificial urinary canal and a second elongate tubular catheter section 2 adapted for manipulation of the catheter. At the proximal end 3, the tubular catheter section is provided with holes 4 enabling urine to drain into the tubular member. In order to protect the mucous membrane, the holes may preferably be provided on the side of the tubular member. Alternatively, a tubular member may be provided with a hole in the tip. It is important that the edge of the hole is rounded smoothly or that the material, for at least this part of the tubular member, is selected with the view not to cut or damage urethra.

At the distal end 5, the tubular member is provided with connecting means 6 for connecting the catheter section to mating connecting means 7 of the second tubular catheter section. Preferably, the first and the second section is made from a thermoplastic elatomeric material, other thermoplastic materials, curable elastomeric materials, polyamide resins or elastomers or any mixture thereof, i.e. the group may comprise materials like, PVC, PU, PE, latex, and/or Kraton™.

FIG. 2 shows a view of the assembled catheter. The second catheter section is adapted to elongate the first catheter section so that the first and the second sections together form a rigid catheter having sufficient length to enable catheterisation. The rigidity of the first section should be sufficient to allow the section to be inserted into urethra without collapsing the section. The second section and the connection 6,7—as shown in FIG. 1—between the second section and first section is provided with a rigidity that allows the insertion of the first section by manipulation of the second section. As seen in FIGS. 1 and 2, the catheter may preferably have gripping means 8 for easing a firm grip and manipulation of the catheter.

As indicated in FIG. 2, the kit may comprise one handle section and a number of catheter sections adapted for insertion or the kit may alternatively be packed in two packages—one containing a handle for multiple use and another separately steriliseable package containing one or more sections adapted for insertion and for one-time use.

FIG. 3 shows a “Swiss-knife” embodiment of the catheter kit. The first catheter section 10 is folded into a slid 11 in the second catheter section 12. The first catheter sections being rotatably hinged to the second catheter section in the hinge connection 13.

FIG. 4 shows the “Swiss-knife” embodiment unfolded. The slid 11 could as an example be covered with a thin latex foil, so as to seal the second catheter section. When the catheter is folded, the first catheter section will simply fold the latex foil radially inwardly into the second catheter section. As the catheter is unfolded, the elasticity and a slight pretension of the foil will lift the foil out of the slit and thereby provide free passage for urine to drain through the second catheter section. The latex foil is not shown in the FIGS. 3 and 4.

FIG. 5 shows an embodiment of the invention wherein a catheter is simply bend, whereby the catheter is divided into a first catheter section 14 and a second catheter section 15 by a collapsed catheter part 16. The catheter is provided with a reinforcement sleeve 17. The connector 18 enables connection of the catheter e.g. to a bag for collecting the urine.

FIG. 6 shows the unfolded catheter of FIG. 5. The sleeve 17 has now been displaced along the catheter so as now to support the catheter around the collapsed part 16 of the catheter.

FIG. 7 shows an embodiment of the catheter kit according to the present invention, wherein the first catheter section, not shown in FIG. 7, is sterilely packed inside the second catheter section 21, the second catheter section being sealed in both ends with sealing caps or foils 22,23.

Preferably the first section is coated with a hydrophilic coating, providing a low friction surface of the first catheter section when treated with a liquid swelling medium. The coating could be of the kind which sustains being activated with the liquid swelling medium for longer time, e.g. for several month. Thereby the liquid swelling medium could be provided in the catheter package from the time of packaging so as to provide a ready-to-use catheter. Hydrophilic coatings are known per se, see e.g. the published patent applications WO 98158988, WO 98/58989, WO 98/58990 or EP 0570370. For this purpose, the sealing caps or foils should preferably be provided in a gas impermeable material for conservation of the humidity and thus the lubricity of the catheter for longer time, e.g. for several month. As an example, the second catheter section and/or the sealing caps may be made from a thermoplastic elatomeric material, other thermoplastic materials, curable elastomeric materials, polyamide resins or elastomers or any mixture thereof, i.e. the group may comprise materials like, PVC, PU, PE, latex, and/or Kraton™. The caps may be provided with a thickness allowing for sufficient gas impermeability. As an alternative, they may be made from metallised foils.

As seen in FIG. 8, the first catheter section is easily withdrawn from the second catheter section by pulling the cap or foil 23 which cap or foil engages the distal end of the first catheter section.

FIG. 9 shows the assembled catheter after the first catheter section has been attached to the second catheter section. The foil or cap 23 can either be removed completely as shown in FIG. 9 or can at least be penetrated by the connecting means 24 of the second catheter section.

FIG. 10 shows an embodiment of the catheter kit wherein the first and the second catheter sections are connected telescopically. The first catheter section is sterilely packed inside the second catheter section 26. The second catheter section being sealed by a first sealing closure 27 and a second sealing closure 28. Prior to use, the first sealing closure is removed. If the first catheter section is provided with a hydrophilic surface layer, and if the catheter section is packed with a liquid swelling medium, the liquid medium may be emptied though the passage opened by the first sealing closure. As best seen in FIG. 11, the second sealing closure engages the first catheter section 30 for easy withdrawal of the first catheter section. When the first catheter section has been completely withdrawn, the distal part of the first catheter section engages the proximal end of the second catheter section in the connecting zone 31 and the second sealing closure easily disengages the first catheter section. The catheter is then in a configuration for use.

FIG. 13 shows a preferred embodiment of the second sealing closure 28, wherein the closure is provided with internal and radially inwardly extending projections 33 adapted for engaging the hole 32 shown in FIG. 12.

FIG. 14 shows another embodiment of the second sealing closure 28, wherein flexible gripping flanges 34 softly grips the proximal (inserted) end of the first catheter section for easy withdrawal of the first catheter section from the second catheter section upon removal of the second sealing closure.

The telescopic embodiment of the catheter kit, disclosed in FIGS. 10-14, should preferably be provided so that the internal diameter of the second catheter section is slightly larger than the external diameter of the first catheter section. This is an advantage, e.g. in the case where the first catheter section is coated with a hydrophilic surface coating and in order not to scrape of the coating when sliding the first catheter section out of the second catheter section. On the other hand, it is an important aspect to provide a connecting zone wherein the first catheter section and the second catheter section firmly engages. Thereby insertion and orientation of the first section is possible merely by manipulation of the second section and without the sections mutually sliding in the telescopic connection. It is furthermore important to assure that the first catheter section does not slip out of the second section in which case the first catheter section might disappear into the urethra. For this purpose, the distal end (opposite the inserted end) of the first catheter section may be provided with a radially outwardly extending flange disallowing the first catheter section to slip out of the second catheter section.

FIG. 15 shows an embodiment of the catheter kit wherein a second catheter section surrounding a first proximal catheter section can be turned inside out thereby the second catheter section protects the first catheter section prior to use. By provision of sealing foils or caps in both ends, the first catheter section may even be kept in a sterile condition inside the second section. Before use, the second catheter section is turned inside out by rolling or curling, whereby the catheter is brought into a configuration for use.

Referring to FIG. 16, one aspect of the present invention relates to a bendable catheter. The catheter is provided e.g. as a soft and flexible plastic hose 35, e.g. at least partly made from a thermoplastic elatomeric material, other thermoplastic materials, curable elastomeric materials, polyamide resins or elastomers or any mixture thereof, i.e. the group may comprise materials like, PVC, PU, PE, latex, and/or Kraton™. The catheter is provided with a zone 36 allowing the catheter to bend. The zone may as an example be formed as a below shaped part of the catheter. If the catheter is relatively long and if a fairly large part of the catheter is to be inserted into the urethra, which is commonly the case for male users, it may be an advantage to provide a catheter which has a bendable zone which on the outside is so smooth that it may be inserted into the urethra. For this purpose the invention relates to a catheter having a supporting member inserted into at least the bendable zone. The supporting member may be a piece of an elongate spring provided with a conduit for draining the urine. The spring will easily provide support for the catheter so that the catheter does not collapse. The spring should be provided with an outer diameter as close to the inner diameter of the catheter hose as possible. As an example, the supporting member may be provided as a small piece of a spring, glued inside the catheter in the zone adapted to be bend. As another example, the supporting member may be provided as a longer spring 37, extending out through the opening of the catheter in the distal end (opposite the inserted end) of the catheter. The supporting member may thereby be removed prior to the insertion of the catheter into the urethra or even simultaneously with the insertion of the catheter into the urethra. For this purpose the supporting member may be provided with a handle 38.

FIG. 17 shows a handle 40 for easy manipulation of the catheter. The handle may be highly appreciated not least for disabled users of the catheter e.g. for people having a reduced dexterity.

FIG. 18 shows a preferred cross-sectional shape of the insertable part of the catheter. As the inserted part has an oval cross-sectional shape, the bending moment around the x-axis (indicated in FIG. 18) will be different from the bending moment around the y-axis. The relatively low bending moment around the y-axis will enhance the ability of the catheter to bend in one direction, and thereby easy the insertion of the catheter past prostate. The relatively high bending moment around the x-axis will enhance the general stiffness of the catheter thereby easy manipulation of the inserted part of the catheter from the part of the catheter not being inserted.

1. A kit for preparing a catheter for draining a human bladder, the kit comprising at least two catheter sections defining a passage therein, the sections being adapted to be arranged in a first mutual configuration wherein the passages are joined into one passage and the sections together constitute a catheter of a length larger than the length of each individual section and having such a rigidity that the catheter is manipulatable by manipulation of at least one of the catheter sections and a second mutual configuration wherein the kit is shorter than when the sections are arranged in the first mutual configuration.

2. A kit according to claim 1, wherein at least two of the catheter sections are comprised in one tubular member, the sections being separated by a structural transition of the tubular member.

3. A kit according to claim 1 or 2, wherein at least one structural transition forms a bendable zone between two adjacent sections of the catheter.

4. A kit according to claim 3, wherein the bendable zone is defined by part of the tubular member having a different bending moment than other parts of the tubular member.

5. A kit according to claim 3 or 4, wherein the bendable zone is defined by a bellow shaped part of the tubular member.

6. A kit according to any of claims 3-5, further comprising a displaceable reinforcement sleeve for supporting the bendable zone of the catheter in the first configuration.

7. A kit according to claim 6, wherein the sleeve is surroundingly arranged on the tubular member.

8. A kit according to claim 7, wherein the sleeve is arranged within the tubular member

9. A kit according to claim 2, wherein at least one structural transition enables the inside of one section to be turned out so as to allow the one section to be wrapped around the other section(s).

10. A kit according to any of the preceding claims, wherein at least two sections are divided into a pair of separate sections joined by connecting means.

11. A kit according to claim 10, wherein the connecting means provides a rigidity allowing for manipulation of at least one of the catheter sections by manipulation of one of the other catheter sections

12. A kit according to claim 10 or 11, wherein the connecting means defines a rotational joint.

13. A kit according to any of claims 10 12, wherein the connecting means defines a telescopical joint.

14. A kit according to any of the preceding claims, wherein at least one of the catheter sections is provided with a hydrophilic surface of at least a part of its surface intended to provide a low-friction surface character of that part of the catheter by treatment with a liquid swelling medium prior to use of the catheter.

15. A kit according to any of the preceding claims, wherein at least one of the catheter sections forms a tube with a cross-sectional flow area the range 0.5 mm²-30 mm², such as in the range of 2 mm²-26 mm²., such as in the range of 5 mm²-22 mm², such as in the range of 10 mm²-16 mm², such as in the range of 12 mm²-14 mm², such as in the size of 13 mm².

16. A kit according to any of the preceding claims, wherein at least one of the catheter sections forms a tube with a hydraulic radius in the range of 0.2-1.5 mm, such as in the range of 0.3-1.4 mm., such as in the range of 0.4-1.3 mm., such as in the range of 0.5-1.2 mm, such as in the range of 0.6-1.1 mm, such as in the range of 0.7-1.0 mm such as in the range of 0.8-0.9 mm.

17. A kit according to any of the preceding claims, wherein at least one of the catheter sections forms a tube with a substantially circular cross sectional shape.

18. A kit according to any of the preceding claims, wherein at least one of the catheter sections forms a tube with a substantially non-circular cross sectional shape.

19. A catheter according to any of the preceding claims, wherein at least one of the catheter sections is provided in a length in the range of 50-90 mm., such as in the range of 55-85 mm., such as in the range of 60-80 mm. such as with a length of 70 mm.

20. A catheter according to any of the preceding claims, wherein at least one of the catheter sections is provided in a length in the range of 180-250 mm., such as in the range of 190-240 mm., such as in the range of 200-230 mm. such as in the size of 220 mm.

21. A kit according to any of the preceding claims, wherein at least one of the sections is provided with a gripping zone for easing the grip during use of the catheter.

22. A kit according to claim 21, wherein the gripping zone is substantially radially extending.

23. A kit according to any of the preceding claims, wherein the sections are arranged in the second configuration within a package.

24. A kit according to claim 23, wherein the package is at least partly formed by at least one of the sections.

25. A kit according to any of claims 23 or 24, wherein the package includes an amount of liquid swelling medium sufficient for the treatment of the hydrophilic surface part to provide a low-friction surface character of that part.

26. A kit according to claim 25, wherein the liquid swelling medium has been brought into contact with the hydrophilic surface for activation of the low-friction surface character before, during or after the arrangement of the catheter sections in the package, so as to provide a ready-to-use catheter.

27. A kit according to any of the preceding claims, wherein at least one of the sections are coated with a lubricant.

28. A kit according to any of the preceding claims, further including a lubricant for coating at least one of the sections of the kit.

29. A kit according to any of claims 23-28, wherein the package includes a lubricant

30. A bendable urinary catheter for draining a human bladder comprising:
a flexible elongated tube with an inner cross-sectional shape and size defining a first conduit for draining urine, said tube having an insertion end and a discharge end, and a supporting member being introduced into the first conduit and provided with an outer cross-sectional shape and size radial size substantially equal to the inner cross-sectional shape and size of the elongate tube so as to support said tube against collapsing during bending of the tube, the supporting member having a flexibility allowing curling.

31. A catheter according to claim 30, wherein the supporting member is hollow and defines a second conduit for draining the urine through the supporting member.

32. A catheter according to claim 30 or 31, wherein the supporting member is provided in a length in the range of 20-60 mm, such as in the range of 30-50 mm., such as in the range of 35-45 mm.

33. A catheter according to claim 30 or 31, wherein the supporting member is provided in a length in the range of 60-120 mm, such as in the range of 70-110 mm., such as in the range of 80-100 mm.

34. A catheter according to claim 30-33, wherein the supporting member is provided in a length in the range of at least one half of the length of the elongated tube.

35. A catheter according to any of claims 30-34, wherein the supporting member is extending out of the discharge end of the elongated tube.

36. A catheter according to claim 35, wherein the supporting member is provided with gripping means for easing withdrawal of the supporting member from the discharge end during insertion of the catheter.

37. A catheter according to any of claims 30-35, wherein the supporting member is an integrated part of the elongated tube.

38. A catheter according to any of the claims 30-37, wherein the supporting member is a spring.

39. A catheter according to any of the claims 30-38, wherein the supporting member is made from a material selected from the group comprising surgical steel, aluminium, a thermoplastic elatomeric material, other thermoplastic materials, curable elastomeric materials, polyamide resins, elastomers and any combination thereof.

40. A supporting member for use with a bendable catheter according to claim 30-39.

41. A catheter section to be introduced into the urethra for draining a bladder, the catheter section being provided with connecting means for connecting the catheter section to a handle.

42. A catheter handle section for introducing a catheter section into the urethra for draining a bladder, the handle section being provided with connecting means for connecting the handle section to a catheter section.
